(19) United States
(12) Patent Application Publication
(10) Pub. No.: US 2023/0241272 A1
(43) Pub. Date: Aug. 3, 2023

(54) A POWERED DISINFECTANT DISPENSING DEVICE

(71) Applicant: Nikeeta Brewton, Warner Robins, GA (US)

(72) Inventor: Nikeeta Brewton, Warner Robins, GA (US)

(21) Appl. No.: 17/588,261

(22) Filed: Jan. 29, 2022

(51) Int. Cl. A61L 2/23; B05B 7/24; B05B 7/00

(52) U.S. Cl. CPC A61L 2/23; B05B 7/2491; B05B 7/0081; A61L 2202/15; A61L 2202/14

(57) ABSTRACT

A powered disinfectant dispensing device for providing disinfectants is presented. The powered disinfectant dispensing device allows for an easy distribution of disinfectant to a person. The device is both light weight and efficient while providing a small footprint for storage. The device automatically dispenses liquid disinfectant in an airborne manner. The device has a fan that is utilized to provide wind behind a disinfectant to cause the disinfectant to become airborne and evenly distribute the disinfectant.

### FIELD OF THE INVENTION

This invention relates to a disinfectant device. More particularly it relates to a powered disinfectant dispensing device.

### BACKGROUND

A disinfectant is a chemical substance or compound used to inactivate or destroy microorganisms on inert surfaces. Disinfection does not necessarily kill all microorganisms, especially resistant bacterial spores; it is less effective than sterilization, which is an extreme physical or chemical process that kills all types of life. Disinfectants are generally distinguished from other antimicrobial agents such as antibiotics, which destroy microorganisms within the body, and antiseptics, which destroy microorganisms on living tissue. Disinfectants are also different from biocides—the latter are intended to destroy all forms of life, not just microorganisms. Disinfectants work by destroying the cell wall of microbes or interfering with their metabolism. It is also a form of decontamination, and can be defined as the process whereby physical or chemical methods are used to reduce the number of pathogenic microorganisms on a surface.

Disinfectants can also be used to destroy microorganisms on the skin and mucous membrane, as in the medical dictionary historically the word simply meant that it destroys microbes. Sanitizers are substances that simultaneously clean and disinfect. Disinfectants kill more germs than sanitizers. Disinfectants are frequently used in hospitals, dental surgeries, kitchens, and bathrooms to kill infectious organisms. Sanitizers are mild compared to disinfectants and are used majorly to clean things that are in human contact whereas disinfectants are concentrated and are used to clean surfaces like floors and building premises. Bacterial endospores are most resistant to disinfectants, but some fungi, viruses and bacteria also possess some resistance.

The term dispenser typically implies a machine or container which is designed to release a specific amount of its content, usually liquids or powders/fine granular materials. Applying disinfectant using a power disinfectant sprayer is more efficient and effective than spray-and-wipe methods. Sprayers provide effective disinfecting by completely coating surfaces to meet required dwell times. Spray-and-wipe methods are not only inefficient, but also ineffective at providing proper dwell times and complete coverage of disinfectant.

Current power disinfectant sprayers are hard to use and can be cumbersome to a person who is distributing the disinfectant. This can cause the person to abandon a more efficient and effective method to distribute the disinfectant.

Accordingly, and in light of the foregoing, there is a need for a device that can distribute the disinfectant that is easily used by a common person. The device should further be light weight and efficient while also having a smaller footprint for storage.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is an isometric view of the powered disinfectant dispensing device 10, according to the preferred embodiment of the present invention;

FIG. 2 is a side view of the powered disinfectant dispensing device 10, according to the preferred embodiment of the present invention;

FIG. 3 is a rear view of the powered disinfectant dispensing device 10, according to the preferred embodiment of the present invention;

FIG. 4 is a sectional view of the powered disinfectant dispensing device 10, as seen along a line I-I, as shown in FIG. 1, according to the preferred embodiment of the present invention;

FIG. 5 is a front view of the first decorative covering 140, as used with the powered disinfectant dispensing device 10, according to the preferred embodiment of the present invention;

FIG. 6 is a front view of the second decorative covering 145, as used with the powered disinfectant dispensing device 10, according to the preferred embodiment of the present invention; and,

FIG. 7 is a front view of the third decorative covering 155, as used with the powered disinfectant dispensing device 10, according to the preferred embodiment of the present invention.

### DESCRIPTIVE KEY

10 powered disinfectant dispensing device
15 inner enclosure
20 formed recess
25 dispersion grill
30 disinfectant
35 mounting receiver
40 generic decorative covering
45 mounting tab
50 cover travel path “c”
55 first energization means
60 wick insertion hole
65 fill spout
70 second energization means
75 release strength adjustment lever
80 battery
85 power supply
90 power wiring
95 motor
100 fan blade
105 wick
110 tube
115 reservoir
120 liquid disinfectant
125 diffusing end
130 level controller
134 level controller path “a”
140 first decorative covering
145 second decorative covering
150 red colored door
155 third decorative covering

### DETAILED DESCRIPTION OF THE DRAWINGS

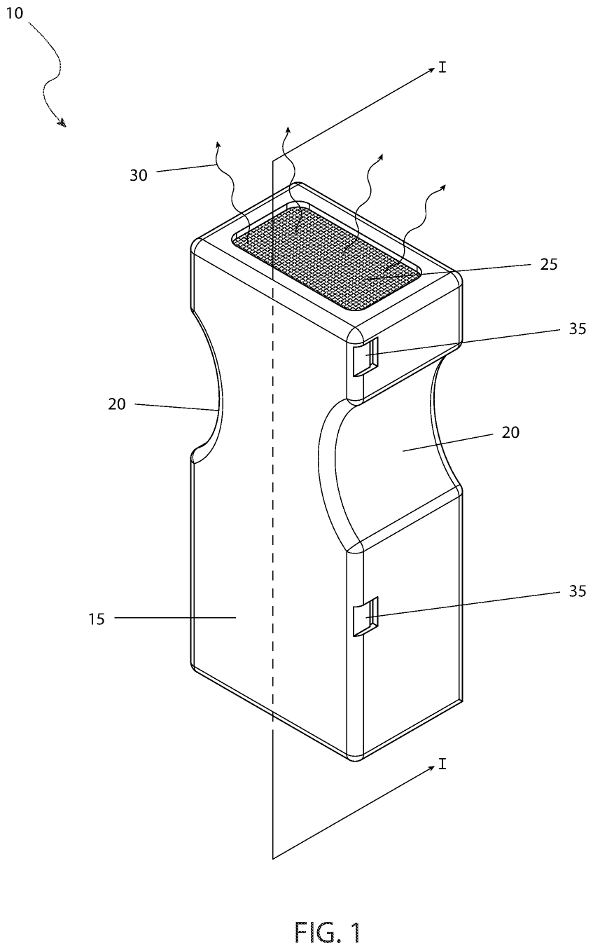

The best mode for carrying out the invention is presented in terms of its preferred embodiment, herein depicted within FIGS. 1 through 7. However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one (1) particular configuration shall be shown and described for purposes of clarity and disclosure and not by way of limitation of scope. All of the implementations described below are exemplary implementations provided to enable persons skilled in the art to make or use the embodiments of the disclosure and are not intended to limit the scope of the disclosure, which is defined by the claims.

The terms “a” and “an” herein do not denote a limitation of quantity, but rather denote the presence of at least one (1) of the referenced items.

Referring now to FIG. 1, an isometric view of the powered disinfectant dispensing device 10, according to the preferred embodiment of the present invention is disclosed. The powered disinfectant dispensing device (herein also described as the “device”) 10, automatically distributes liquid disinfectant 120 in an airborne manner. The device 10 provides for an inner enclosure 15 made of plastic. The overall dimensions of the inner enclosure 15 are approximately three inches (3 in.) wide, six inches (6 in.) tall and one inch (1 in.) deep. The inner enclosure 15 is provided with formed recesses 20 on either side that function as hand grips to allow for easy handling of the inner enclosure 15. The top of the inner enclosure 15 is provided with a dispersion grill 25 that allows for release of atomized disinfectant 30. Operation and functionality of the release of the atomized disinfectant 30 will be described in greater detail herein below. The inner enclosure 15 is provided with a total of four (4) mounting receivers 35 (of which only two (2) are shown due to illustrative limitations), two (2) per each side of the inner enclosure 15, that provide for the securement of a generic decorative covering 40 (not shown in this figure). Further description of the usage and operation of the generic decorative covering 40 will be described herein below.

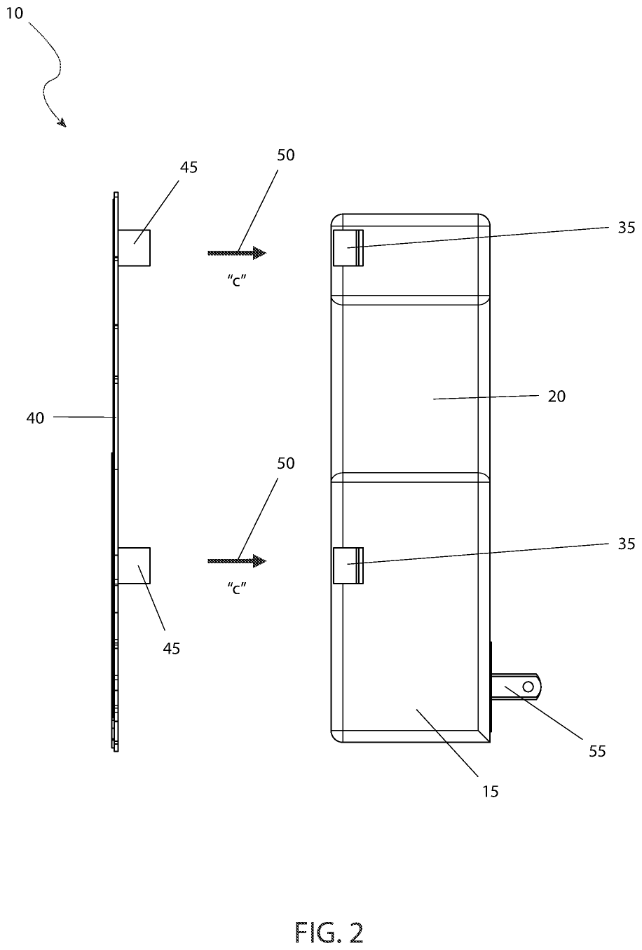

Referring next to FIG. 2, a side view of the device 10, according to the preferred embodiment of the present invention is depicted. The side of the inner enclosure 15 depicts the formed recesses 20 as well as the mounting receivers 35 as aforementioned described. The generic decorative covering 40 is provided with four (4) mounting tabs 45 (two (2) (per side) (of which only two (2) are shown due to illustrative limitations). The generic decorative covering 40 is moved along a cover travel path “c” 50, such that the mounting tabs 45 engage and hold against the mounting receivers 35 using mechanical friction fit. A first energization means 55 herein depicted as AC power plug is shown protruding from the rear of the inner enclosure 15. Such first energization means 55 allows not only for energization of the device 10, but also provides for a physical mounting means at any AC electrical outlet.

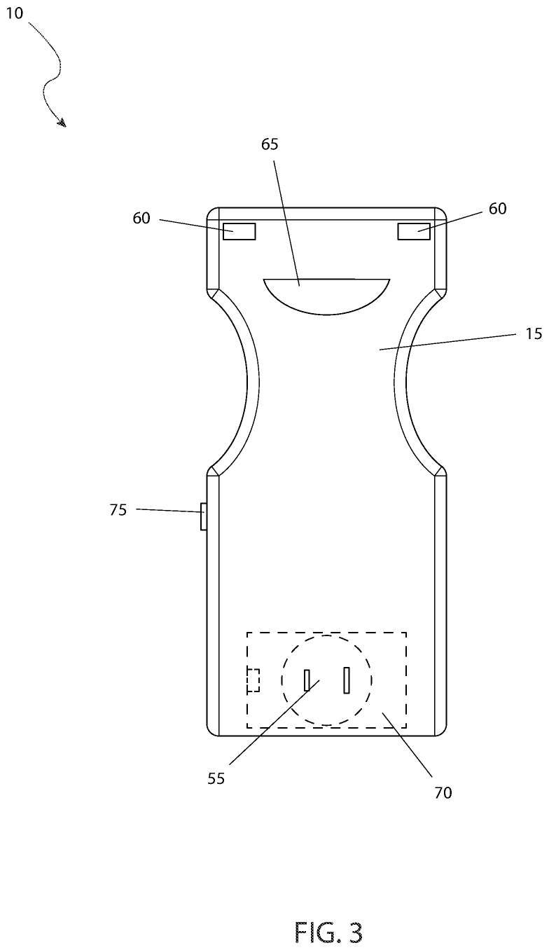

Referring now to FIG. 3, a rear view of the device 10, according to the preferred embodiment of the present invention is shown. The rear of the inner enclosure 15 discloses two (2) wick insertion holes 60 at the top of the inner enclosure 15. Further description and utilization of the wick insertion holes 60 will be described herein below. A fill spout 65 is located immediately below the wick insertion holes 60 which is utilized to fill the device 10 with liquid disinfectant 120. The lower portion of the inner enclosure 15 is provided with the first energization means 55 or a second energization means 70 such as battery compartment. The second energization means 70 allows for use of the device 10 with batteries to allow for portable operation anywhere without reliance on an AC power outlet as is the case with the first energization means 55. It is envisioned that the device 10 would be equipped with either the first energization means 55 or the second energization means 70. The first energization means 55 allows for continuous operation without worry of battery replacement nor a dedicated mounting space. The second energization means 70 allows for portable operation that can be placed anywhere without reliance on AC power. A release strength adjustment lever 75 is visible on the side of the inner enclosure 15 to allow for adjustment of the amount or power of the atomized disinfectant 30 (as shown in FIG. 1). Further description of the release strength adjustment lever 75 will be provided herein below.

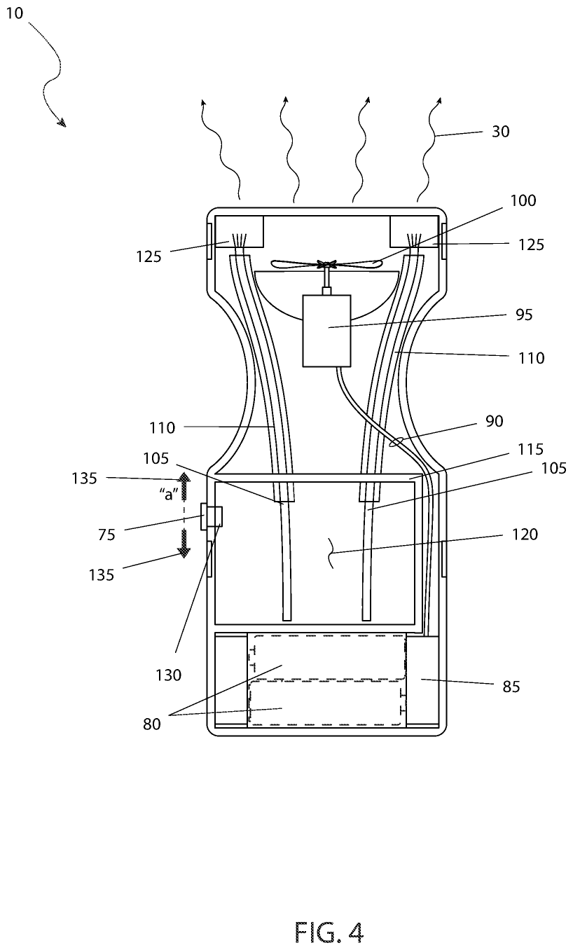

Referring next to FIG. 4, a sectional view of the device 10, as seen along a line I-I, as shown in FIG. 1, according to the preferred embodiment of the present invention is disclosed. The lower portion of the inner enclosure 15 discloses batteries 80 that would be used in the case of a device 10 powered by a second energization means 70 (as shown in FIG. 3). A power supply 85 would be used in the case of a device 10 powered by a first energization means 55 (as shown in FIG. 2). In either case, power wiring 90 carries electric power to a motor 95 which operates a fan blade 100. Two (2) wick(s) 105 are inserted through the wick insertion holes 60 (as shown in FIG. 3) and into two (2) separate tubes 110 which route the wicks 105 into a reservoir 115 holding liquid disinfectant 120. The wicks 105 are designed to be user replaceable via the wick insertion holes 60 over the useful lifetime of the device 10. The upper portion of each wick 105 is provided with a diffusing end 125 that increases the surface area of the wick 105 to allow for ease of evaporation and diffusion. The release strength adjustment lever 75 is connected to a level controller 130 which travels along a level controller path “a” 135. The level controller 130 provides for more (i.e., further insertion of the wicks 105 into the reservoir 115) or less (i.e., reduced insertion of the wicks 105 into the reservoir 115) for more or less liquid disinfectant 120 that is dispersed as atomized disinfectant 30 through the dispersion grill 25 (as shown in FIG. 1). Thus, as the fan blade 100 rotates, air flow causes the liquid disinfectant 120 to be released from the diffusing end 125 of the wicks 105 and generates an atomized disinfectant 30 which is dispersed to the living space, the device 10 is located in.

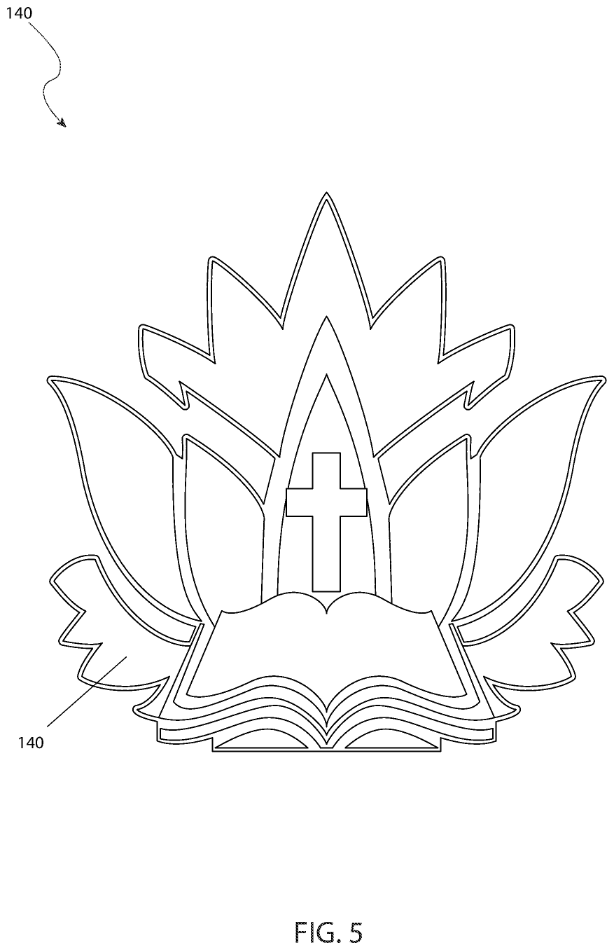

Referring now to FIG. 5, a front view of the first decorative covering 140, as used with the device 10, according to the preferred embodiment of the present invention is depicted. The first decorative covering 140 is depicted as a pictorial representation of a healing temple. The first decorative covering 140 provides an aesthetically pleasing appearance to the front view of the device 10.

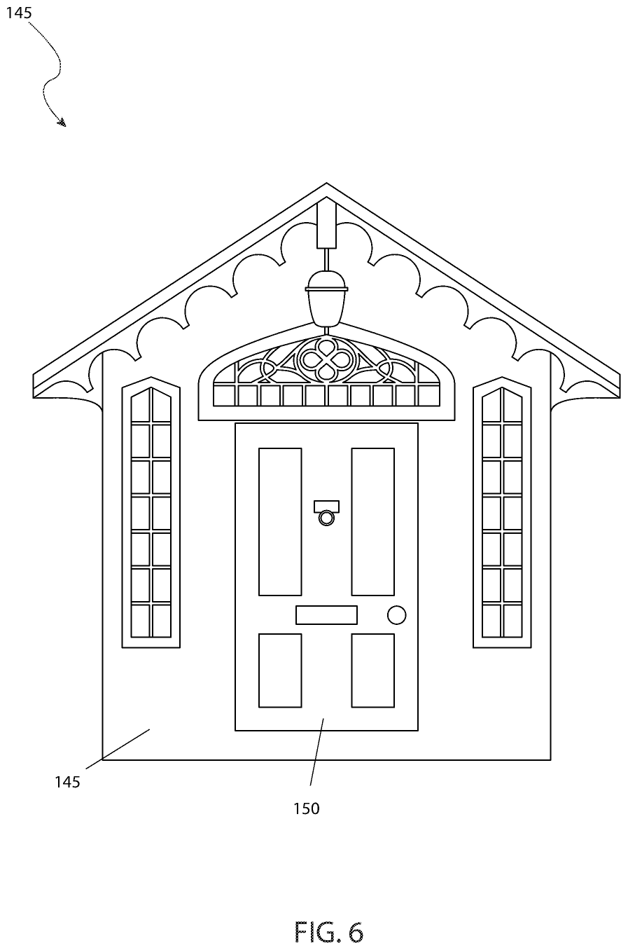

Referring next to FIG. 6, a front view of the second decorative covering 145, as used with the device 10, according to the preferred embodiment of the present invention is shown. The second decorative covering 145 is depicted as a pictorial representation of a red door house complete with a red-colored door 150. The second decorative covering 145 provides another aesthetically pleasing appearance to the front view of the device 10.

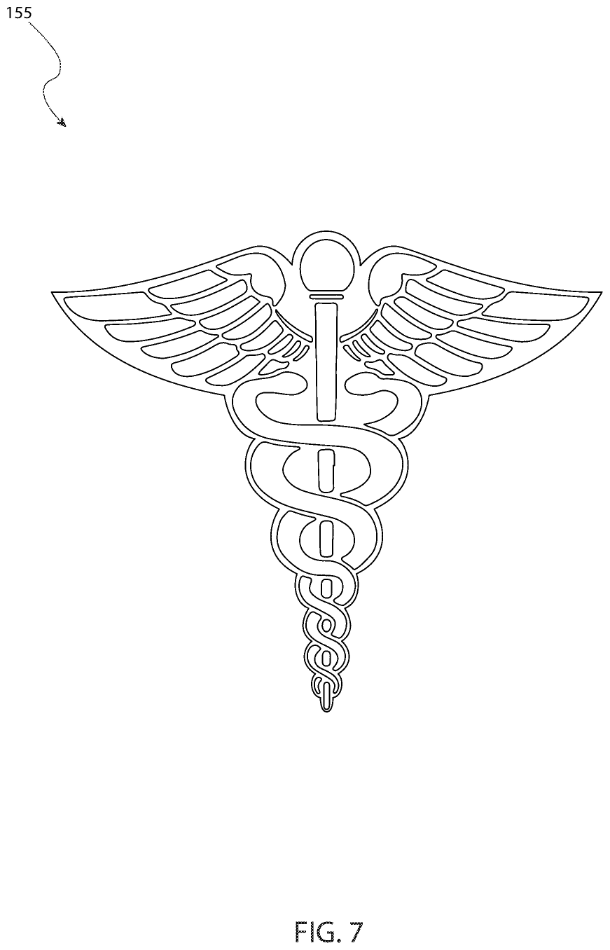

Referring to FIG. 7, a front view of the third decorative covering 155, as used with the device 10, according to the preferred embodiment of the present invention is disclosed. The third decorative covering 155 is depicted as a pictorial representation of a medical sign. The third decorative covering 155 provides yet another aesthetically pleasing appearance to the front view of the device 10.

### Operation of the Preferred Embodiment

The preferred embodiment of the present invention can be utilized by the common user in a simple and effortless manner with little or no training. It is envisioned that the device 10 would be constructed in general accordance with FIG. 1 through FIG. 7. The user would procure the device 10 from conventional procurement channels such as discount stores, department stores, drug stores, hardware stores, home improvement stores, mechanical supply houses, mail order and interne supply houses and the like.

After procurement and prior to utilization, the device 10 would be prepared in the following manner: the user would insert two (2) wicks 105 through the wick insertion holes 60, using a funnel or other filling aid; liquid disinfectant 120 is poured through the fill spout 65 and into the reservoir 115; and an appropriate dispersion level is selected via the release strength adjustment lever 75 which mechanically operates the level controller 130. Should the device 10 be provided with a first energization means 55, it is connected to a conventional AC power outlet. Should the device 10 be provided with a second energization means 70, batteries 80 are inserted into the inner enclosure 15; the user would apply either the first decorative covering 140, the second decorative covering 145, or the third decorative covering 155 using the mounting tabs 45 and engaging the mounting receivers 35 on the inner enclosure 15. At this point in time, the device 10 is ready for operation.

During utilization of the device 10, the device 10 operates in a transparent manner, without any user interface. As the fan blade 100 rotates as powered by the motor 95, air flow causes the liquid disinfectant 120 to be released from the diffusing end 125 of the wicks 105 thus generating the atomized disinfectant 30 which is dispersed to the living space, the device 10 is located in. Such action continues until the reservoir 115 is depleted, or the user stops operation of the motor 95 by unplugging the device 10 from an AC power outlet in the case of a device 10 operated by a first energization means 55 or removing the batteries 80 in the case of a device 10 operated by a second energization means 70.

Features of the device 10 are envisioned to be beneficial for times of pandemics such as COVID-19, for user suffering from respiratory illnesses and diseases, asthma, Chronic obstructive pulmonary disease, or COPD, pneumonia, allergies, and the like.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

What is claimed is:

1. A powered disinfectant dispensing device for dispensing a disinfectant, the device comprises:
an inner enclosure, the inner enclosure comprising;
a plurality of sides; and
one or more formed recess, each of the formed recesses being coupled to one of the plurality of sides of the inner enclosure;
a top, the top of the inner enclosure having a dispersion grill;
a plurality of mounting receivers, one or more of the plurality of mounting receivers coupled to the sides of the inner enclosure; and
a reservoir, the reservoir having one or more tubes, the tubes being coupled to one of the wick insertion holes, wherein the reservoir having a liquid disinfectant;
a generic covering, the generic covering comprising:
a plurality of sides; and
a plurality of mounting tabs, one or more of the mounting tabs being coupled to one of the sides of the generic covering, wherein each of the mounting tabs being coupled to one of the mounting receivers of the inner enclosure;
a rear, the rear having a plurality of wick insertion holes;
a reservoir, the reservoir for holding a disinfectant;
a plurality of wick insertion holes configured on a rear of the generic covering;
a plurality of tubes, each of the tunes being coupled to one of the wick insertion holes and to the reservoir;
a plurality of wicks, the wicks being configured to be inserted through the wick insertion holes, the wicks being inserted through wick insertion holes and through the tubes and into the reservoir;
a fill spout, the fill spout being on a rear of the generic covering configured below at least one of the wick insertion holes;
an energization means;
a power source;
a wiring being coupled to the power source;
a motor, the motor being coupled to the power source by the wiring; and
a fan blade, the fan blade being operated by the motor;
a level controller, the level controller configured for further insertion of wicks into the reservoir; and
a release strength adjustment lever coupled to the level controller being configured to actuate the level controllers.

2. The device of claim 1, wherein the power source is a battery.

3. The device of claim 1, wherein the power source is an AC outlet.

4. The device of claim 1, wherein the mounting receivers are four (4) in number.

5. The device of claim 1, wherein the inner enclosure is three (3) inches in width.

6. The device of claim 1, wherein the inner enclosure is six (6) inches in height.

7. The device of claim 1, wherein the inner enclosure is one (1) inch in depth.

8. The device of claim 1, wherein the mounting receivers are two (2) on each of the sides.

9. The device of claim 1, wherein the wicks are two (2) in number.

10. The device of claim 1, wherein the wicks have a first end and a second end, the first end of each of the wicks having a diffusing end, and the second end of each of the wicks being configured to be in the reservoir.
